(19) United States
(12) Patent Application Publication
(10) Pub. No.: US 2006/0196501 A1
(43) Pub. Date: Sep. 7, 2006

(54) Systems and methods for mixing and sparging solutions and/or suspensions

(75) Inventors: Kenneth L. Bibbo, Freehold, NJ (US); Mark A. Canter, Raleigh, NC (US); Michael E. Goodwin, Logan, UT (US); Jeremy K. Larsen, Providence, UT (US); Nephi D. Jones, Newton, UT (US)

Correspondence Address: WORKMAN NYDEGGER;(F/K/A WORKMAN NYDEGGER & SEELEY), 60 EAST SOUTH TEMPLE, 1000 EAGLE GATE TOWER, SALT LAKE CITY, UT, 84111, US

(73) Assignee: Hynetics LLC

(21) Appl. No.: 11/127,651

(22) Filed: May 11, 2005

Related U.S. Application Data
(60) Provisional application No. 60/657,824, filed on Mar. 2, 2005.

(51) Int. Cl. A61M 11/00

(52) U.S. Cl. 128/200.23

(57) ABSTRACT

A system for mixing a liquid solution or suspension includes a collapsible container having an interior surface bounding a compartment, the compartment being adapted to hold a liquid solution or suspension. A mixer is enclosed within the compartment of the container. A shaft has a first end connected to the mixer within the compartment of the container and a second end disposed outside of the compartment, the shaft being selectively movable relative to the container. A gas pathway extends from a portion of the shaft disposed outside of the container to a portion of the shaft or mixer disposed within the compartment of the container, the gas pathway extending through at least a portion of the shaft.

### CROSS-REFERENCE TO RELATED APPLICATIONS

This application claims priority to U.S. Provisional Application Ser. No. 60/657,824, filed Mar. 2, 2005, which is incorporated herein by specific reference.

### BACKGROUND OF THE INVENTION

1. The Field of the Invention

The present invention relates to systems and methods for mixing and sparging solutions and/or suspensions such as cultures of cells or microorganisms.

2. The Relevant Technology

Bioreactors are used in the growth of cells and microorganisms. Conventional bioreactors comprise a rigid tank that can be sealed closed. A drive shaft with propeller is rotatably disposed within the tank. The propeller functions to suspend and mix the culture. A sparger is mounted on the bottom of the tank and is used to deliver gas to the culture to control the oxygen content and pH of the culture.

Great care must be taken to sterilize and maintain the sterility of the bioreactor so that the culture does not become contaminated. Accordingly, between the production of different batches of cultures, the mixing tank, mixer, and all other reusable components that contact the culture must be carefully cleaned to avoid any cross contamination. The cleaning of the structural components is labor intensive, time consuming, and costly. For example, the cleaning can require the use of chemical cleaners such as sodium hydroxide and may require steam sterilization as well. The use of chemical cleaners has the additional challenge of being relatively dangerous to use and cleaning agents can be difficult and/or expensive to dispose of once used.

In addition to being labor intensive to clean, conventional bioreactors have operational shortcoming. For example, mixing can be a critical element in a bioreactor. The mixing must be sufficient to uniformly suspend the cells and must effectively disperse the sparged gas into the culture. However, the mixing and sparging must not be so aggressive as to damage the cells or produce excess foam. Although propellers produce satisfactory mixing at some bioreactor scales, they have difficulty in optimizing the desired properties at different scaled sizes.

In addition, conventional bioreactors require a separate sparger that must be placed at the bottom the tank. Such spargers can be difficult to properly place and maintain while retaining the desired sterility. Likewise, the spargers can obstruct uniform mixing and suspension of the culture within the tank. In addition, conventional spargers are difficult to clean and can impact the cleaning of other items within the tank.

Accordingly, what is needed are bioreactors that are efficient to operate, require minimal sterilization, and minimize or eliminate cleaning. Bioreactors are also needed which maximize mixing and suspension while minimizing any shear force applied to the culture. Furthermore, bioreactors are need that are easily scalable while minimizing the formation of foam. What is also need are bioreactors that optimize sparging while eliminating the problems associated with conventional spargers.

### BRIEF DESCRIPTION OF THE DRAWINGS

Various embodiments of the present invention will now be discussed with reference to the appended drawings. It is appreciated that these drawings depict only typical embodiments of the invention and are therefore not to be considered limiting of its scope.

FIG. 1 is an elevated front view of one embodiment of a system for mixing and a sparging a solution and/or suspension;

FIG. 2 is a cross sectional top view of the housing of the system shown in FIG. 1 taken along section lines 2-2;

FIG. 2A is an enlarged section view of the housing shown in FIG. 2;

FIG. 3 is a partially cut away side view of the side wall of the housing shown in FIG. 1 illustrating fluid channels therein;

FIG. 4 is a cross sectional side view of the housing taken along section lines 4-4 of FIG. 2;

FIG. 5 is an elevated front view of an alternative embodiment of a housing;

FIG. 6 is a top plan view of the housing shown in FIG. 5;

FIG. 7 is a cross sectional side view of a container of the system shown in FIG. 1 housing a mixer;

FIG. 8A is a cross sectional side view of the mixer and mixing shaft shown in FIG. 7 with a diaphragm in a first position;

FIG. 8B is a cross sectional side view of the mixer and mixing shaft shown in FIG. 8A with the diaphragm in a second position;

FIG. 9 is a top plan view of the mixer shown in FIG. 8A;

FIG. 10 is a bottom plan view of the mixer shown in FIG. 8A with the flaps thereof in a closed position;

FIG. 11 is a bottom perspective view of the mixer shown in FIG. 10 with the flaps in an open position;

FIG. 12 is an enlarged cross sectional side view of the mixing shaft coupled with the diaphragm; and

FIG. 13 is a cross sectional side view of the mixer and mixing shaft shown in FIG. 8A having an alternative gas pathway.

### DETAILED DESCRIPTION OF THE PREFERRED EMBODIMENTS

The present invention relates to systems and methods for mixing and sparging solutions and/or suspensions. The systems can be commonly used as bioreactors for culturing cells and microorganisms. By way of example and not by limitation, the inventive systems can be used in culturing bacteria, fungi, algae, plant cells, animal cells, protozoans, nematodes, and the like. The systems can accommodate cells and microorganisms that are aerobic or anaerobic and are adherent or non-adherent. The systems can also be used in association with the formation and/or treatment of solutions and/or suspensions that are not biological but nevertheless incorporate mixing and sparging. For example, the systems can be used in the formation of media where sparging is used to control the pH of the media through adjustment of the carbonate/bicarbonate levels with controlled gaseous levels of carbon dioxide.

In one embodiment of the present invention, the systems can be designed so that the container holding the solution and/or suspension and the mixer are disposable. This enables an easy transition between different batches and eliminates many of the problems associated with traditional aseptic systems. The present invention also provides unique mixing and sparging of solutions and/or suspensions so that the growth of the cells and microorganisms can be optimized.

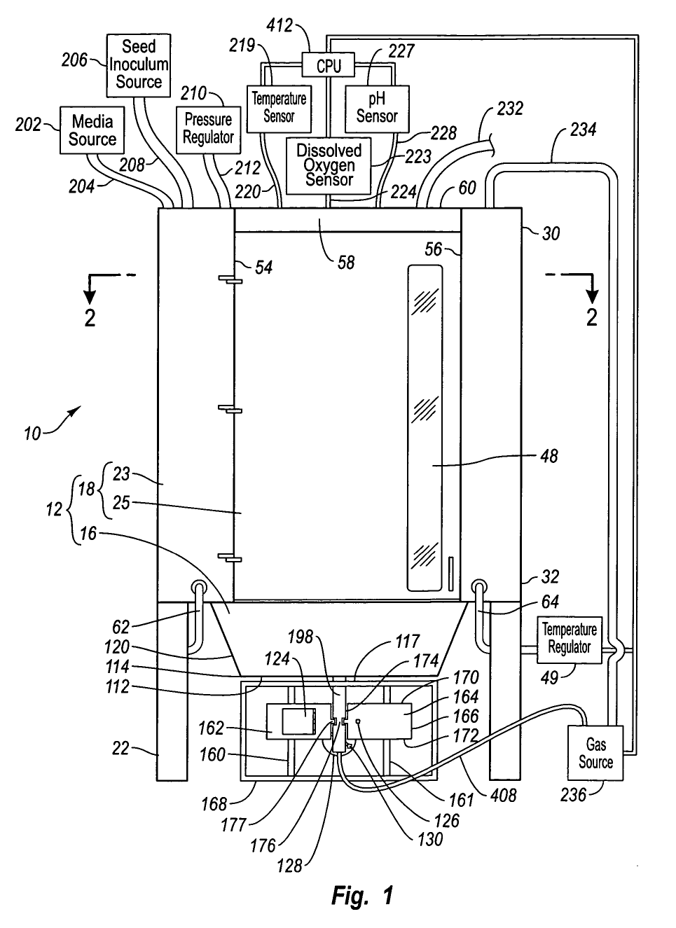

Depicted in FIG. 1 is one embodiment of a system 10 incorporating features of the present invention. System 10 is discussed below in terms of functioning as a bioreactor. In alternative embodiments, however, system 10, either in its present form or in a modified form, can function to mix and sparge solutions and/or suspensions that are not biological. System 10 generally comprises a rigid housing 12 that supports a flexible container 14 (FIG. 6). Container 14 houses a culture that comprises a growth media and cells or microorganisms during the culturing process. Housing 12 comprises a floor 16 and an annular side wall 18 upstanding therefrom. In the embodiment depicted, a plurality of legs 22 extend from side wall 18 to support housing 12 off of a floor or other resting surface. In alternative embodiments, other types of support structures can also be used to support housing 12.

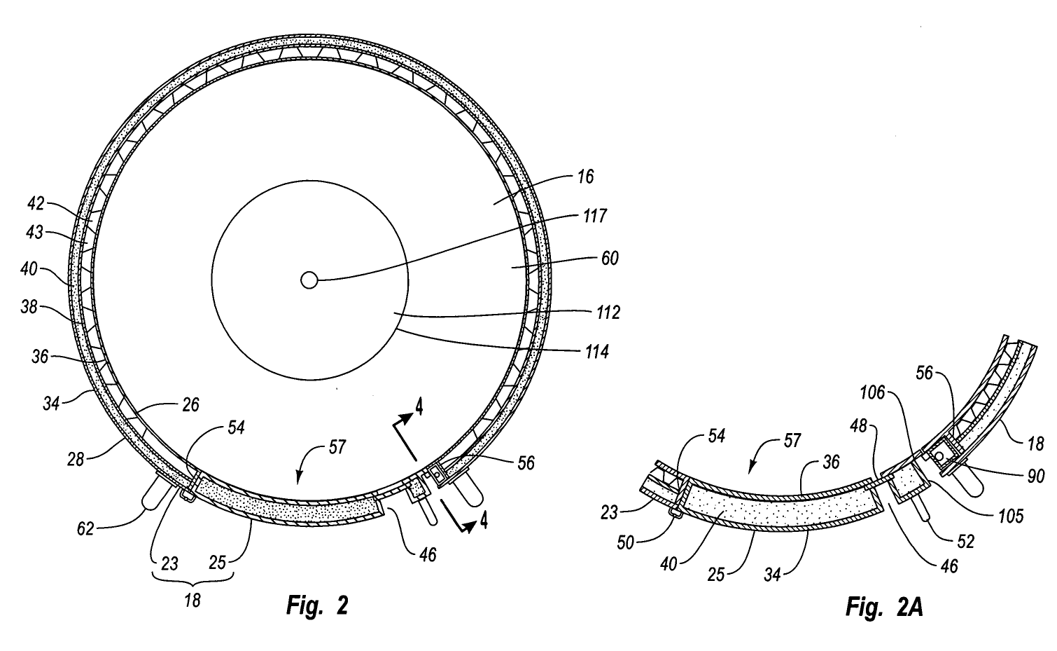

Side wall 18 is jacketed to facilitate temperature control of the culture within container 14. Specifically, as shown in FIGS. 1 and 2, side wall 18 has an interior surface 26 and an exterior surface 28 each extending between an upper end 30 and an opposing lower end 32. Interior surface 26 bounds a chamber 60 that is open at upper end 30. Side wall 18 comprises a body portion 23 having a substantially C-shaped transverse cross section and a door 25. Body portion 23 terminates at substantially opposingly facing end plates 54 and 56 with a doorway 57 formed therebetween. Although not required, to increase the hoop strength of body portion 23, a support brace 58 rigidly extends between end plates 54 and 56 at upper end 30.

Depicted in FIG. 2, body portion 23 comprises an outer wall 34, a concentrically disposed inner wall 36, and a central wall 38 concentrically disposed between outer wall 34 and inner wall 36. Each of walls 34, 36, and 38 connect with each of end plates 54 and 56. Disposed between outer wall 34 and central wall 38 is an insulation layer 40. In one embodiment, insulation layer 40 comprises a chloride free, ceramic fiber capable of withstanding temperatures up to 1,300° C. Other conventional types of insulation can also be used. A sealed fluid compartment 43 is formed between inner wall 36 and central wall 38. A plurality of spaced apart spacers 42 extend between central wall 38 and inner wall 36 across fluid compartment 43. Spacers 42 can comprise discrete members or formations projecting from central wall 38 and or inner wall 36. Spacers 42 provide structural stability for both central wall 38 and inner wall 36 while allowing fluid to freely flow therearound. Although spacers 42 can be other desired configurations, in the embodiment depicted spacers 42 have a frustoconical configuration.

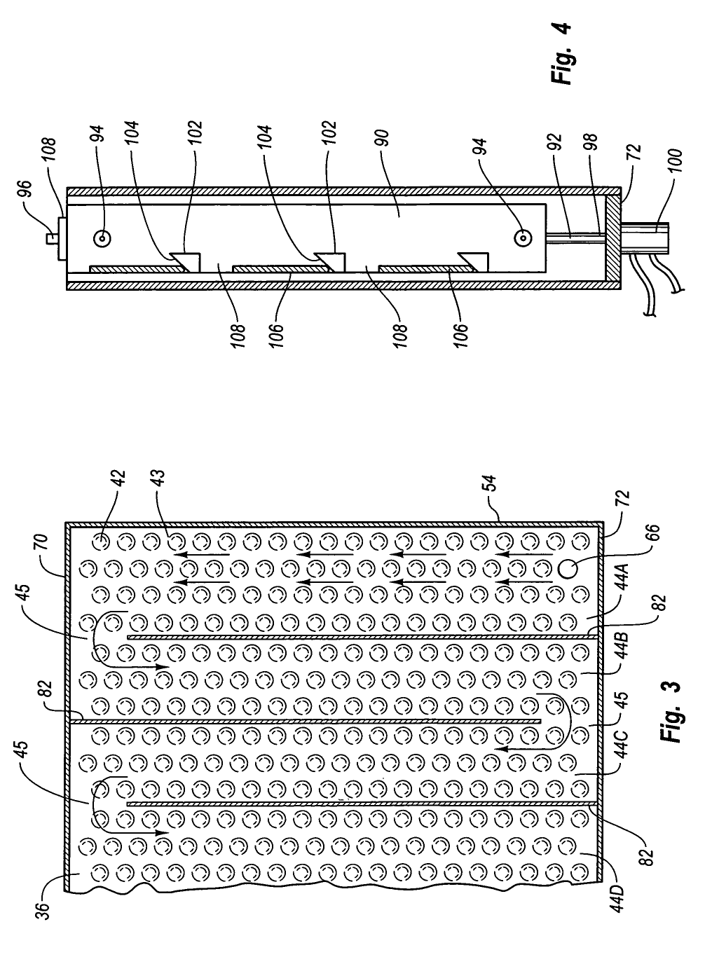

Depicted in FIG. 3 is a cutaway view showing the outside face of inner wall 36 with spacers 42 projecting therefrom. Each of inner wall 36, central wall 38, and outer wall 34 extend between and rigidly connect with a top plate 70 and an opposing bottom plate 72. In one embodiment, support brace 58, previously discussed with FIG. 1, can be integrally formed with top plate 70. A plurality of spaced apart, vertically oriented channeling ribs 82 extend between inner wall 36 and central wall 38 so as to divide fluid compartment 43 into a series of vertical fluid channels 44A, 44B . . . . In alternating ribs 82, a gap 45 is formed between the corresponding channeling rib 82 and top plate 70 or base plate 72 so that the fluid can flow through gap 45. Channeling ribs 82 are thus positioned such that as fluid flows radially about body portion 23, the fluid is also forced to flow in a sinusoidal path along the height of body portion 23.

Specifically, as depicted in FIGS. 1 and 2, a fluid inlet pipe 62 is connected with body portion 23 at lower end 32 adjacent to end plate 54 while a fluid outlet pipe 64 is connected with body portion 23 at lower end 32 adjacent to end plate 56. Each of inlet pipe 62 and outlet pipe 64 are in fluid communication with fluid compartment 43. As fluid is pumped into fluid inlet pipe 62, the fluid enters a first fluid channel 44A through an inlet port 66 shown in FIG. 3. As a result of being bounded between end plate 54 and channeling rib 82, the fluid travels vertically upward where it then passes though gap 45 and then down second fluid channel 44B. This process is repeated around body portion 23 until the fluid exits through outlet pipe 64. Once the fluid is removed, the fluid is then heated or cooled through a temperature regulator 49 (FIG. 1), depending on desired operating parameters, and then reintroduced back through fluid inlet pipe 62. In one embodiment, the fluid passing through fluid compartment 43 is a mixture of water and propylene glycol. In other embodiments, the fluid can be any material that can be used for heating and/or cooling.

In one embodiment of the present invention, means are provided for selectively heating or cooling the solution or suspension held within chamber 60 of housing 12. One example of such means comprises fluid compartment 43 and related structure as discussed above. By running a fluid through fluid compartment 43 with the fluid at a desired temperature, the fluid acts as either a heat sink by drawing energy from the culture through inner wall 36 or as a heat source by inputting energy into the culture through inner wall 36, thereby heating or cooling the culture.

In part, channeling ribs 82 function to uniformly distribute the fluid over the exterior surface of inner wall 36 so as to uniformly control the temperature of the culture within chamber 60. In this regard, channeling ribs 82 and fluid channels 44 can be oriented to flow in a variety of different paths. Furthermore, body portion 32 can be formed without channeling ribs 82.

In yet other alternative embodiments for the means for selectively heating and cooling, fluid compartment 43 can be replaced with piping that runs on the interior, exterior, and/or within inner wall 36. The piping is configured to have the heating or cooling fluid run therethrough. Electrical heating elements, such as resistance heaters, can also be positioned on the interior, exterior, and/or within the inner wall 36 to facilitate heating of culture within chamber 60. In this embodiment, side wall 18 need not be jacketed. In yet another embodiment, the culture within chamber 60 can be pumped out of chamber 60 where it is then selectively heated or cooled through conventional systems and then cycled back into chamber 60.

As depicted in FIG. 2A, side wall 18 also comprises a door 25 disposed within doorway 57 between end plates 54 and 56. As with body portion 23, door 25 comprises an outer wall 34 and an inner wall 36. In this embodiment, however, door 25 does not include a central wall 38. Rather, a layer of insulation 40 is disposed between walls 34 and 36. In an alternative embodiment, door 25 can also include fluid channels 44 which communicate with body portion 23 through flexible hose connections.

A vertically oriented, elongated viewing slot 46 extends through a portion of door 25. A window 48 is disposed within viewing slot 46 so as to seal viewing slot 46 closed but provide an unobstructed view of chamber 60. Door 25 is mounted to body portion 23 by hinges 50. A handle 52 is formed on door 25 to facilitate hinged movement of door 25 between an open position (not shown) wherein free access is provided to chamber 60 through open doorway 57 and a closed position wherein door 25 closes off doorway 57.

In one embodiment of the present invention, means are provided for selectively locking door 25 in the closed position. By way of Example and not by limitation, as depicted in FIGS. 2A and 4, a vertically oriented, tubular housing 90 is movable mounted along end plate 56 of body portion 23. Housing 90 has a front face with a plurality of vertically spaced apart stops 102 formed thereon. Each stop 102 has an engagement face 104 that slopes toward chamber 60.

An actuation rod 92 extends through housing 90 in parallel alignment therewith. Actuation rod 92 is rigidly secured to housing 90 by bolts 94 or the like and extends between a first end 96 and an opposing second end 98. First end 96 of actuation rod 92 projects up above tubular housing 90. Second end 98 of actuation rod 92 is coupled with a hydraulic piston 100 disposed below bottom plate 72. By selectively operating hydraulic piston 100, actuation rod 92 is selectively raised and lowered which in turn selectively raises and lowers housing 90.

Projecting from a side face 105 of door 25 is a plurality of vertically oriented and spaced apart locking flanges 106 (FIG. 2A). Each locking flange 106 is separated by a gap 108. To facilitate locking of door 25, actuation rod 92 is moved to a lowered position and door 25 is moved to the closed position. In this configuration, locking flanges 106 are disposed between stops 102. Hydraulic piston 100 is then used to elevate actuation rod 92. In so doing, housing 90 and stops 102 rise so that engagement face 104 of each stop 102 biases against a corresponding locking flange 106. Engagement faces 104 are sloped so as to bias locking flanges 106 radially inward, thereby locking door 25 closed. To further secure this locking, a hole can be formed in support brace 58 (FIG. 1) in alignment with actuation rod 92. As actuation rod 92 rises, first end 96 of actuation rod 92 passes through the hole in support brace 58.

It is appreciated that the means for selectively locking door 25 can have a variety of alternative configurations. By way of example and not by limitation, hydraulic piston 100 can be replaced by a pneumatic piston, gear or belt drive, crank, jack, or other drive mechanism. Furthermore, is appreciated that locking flanges 106 and stops 102 can be switched or replaced with a variety of other conventional interlocking members. In other embodiments, a variety of shafts can be positioned so as to selectively drive from one of door 25 or body portion 23 into or against the other thereof. Hand operated dead bolts and other conventional locking structures can also be used.

Returning back to FIGS. 1 and 2, floor 16 is rigidly connected to side wall 18. Floor 16 comprises a substantially flat base floor 112. In the embodiment depicted, base floor 112 is circular and extends to a perimeter edge 114. As will be discussed below in greater detail, a central access hole 117 extends through base floor 112. Although not required, a plurality of screened spill holes can also formed on base floor 112.

A peripheral wall 120 upwardly and outwardly slopes from perimeter edge 114 of base floor 112 to side wall 18. Floor 16 and the walls of side wall 18 are typically made of a metal such as stainless steel. In the embodiment depicted, floor 16 has a substantially frustoconical configuration. In alternative embodiments, floor 16 can be entirely flat, curved, pyramidal, conical, or any other desired configuration that can support a bag as discussed below. Furthermore, floor 16 need not be circular but can be polygonal, elliptical, irregular, or any other desired configuration.

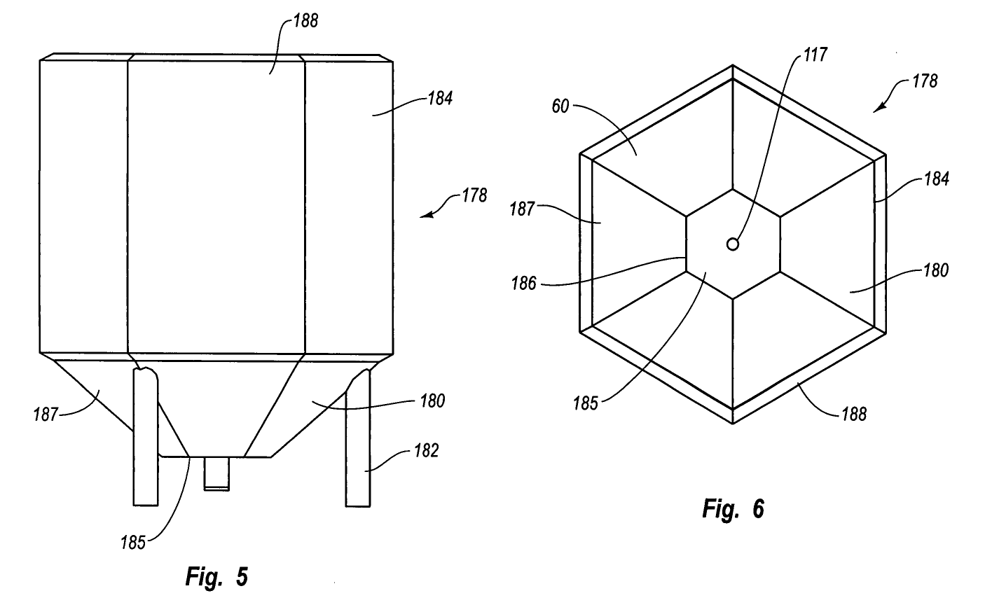

It is appreciated that housing 12 can have a variety of different configurations and designs. For example, depicted in FIGS. 5 and 6 is a housing 178 comprising a substantially frustoconical floor 180 having a plurality of support legs 182 downwardly extending therefrom. Rigidly connected to and upwardly extending from the perimeter of floor 180 is an annular side wall 184. Floor 180 and side wall 184 bound chamber 60.

Floor 180 comprises a central base floor 185 having central access hole 117 extending therethrough. Base floor 185 has a hexagonal configuration that terminates at a plurality of perimeter edges 186. A trapezoidal shaped floor panel 187 upwardly extends at an angle from each perimeter edge 186 of base floor 185. Each of floor panels 187 is secured, such as by welding, bolting, or the like, to the adjacent floor panels 187. The resulting floor 185 thus has a substantially frustoconical configuration with an interior surface, an exterior surface, and a perimeter edge each having a substantially hexagonal transverse cross section.

Side wall 184 comprises a plurality of side panels 188 each having a substantially rectangular configuration. Each side panel 188 is rigidly connected to and upwardly extends from an outer perimeter edge of a corresponding floor panel 187. Again, adjacent side panels 188 are connected to each other and to floor panels 187 such as by welding, bolting, or the like. Side wall 184 thus has an interior surface and an exterior surface each having a substantially hexagonal transverse cross section along the length of side wall 184. In further contrast to housing 20, side wall 184 does not include a door or window, although any number of doors and windows can be formed.

In both housing 20 and housing 178, the side wall and floor can be any desired configuration such as elliptical, polygonal, irregular, or any other desired configuration. The floor typically has a configuration complementary to the side wall. In alternative embodiments, it is appreciated that the various features of housings 20 and 178 can be mixed and matched so as to produce a variety of housing configurations having different properties. Furthermore, housings can be made in any number of different sizes. For example, housings can be made with a chamber 60 having a volume of 30 liters, 100 liters, 250 liters, 500 liters, 750 liters, 1,000 liters, 1,500 liters, 3,000 liters, 5,000 liters, 10,000 liters or other sizes. Other examples of housings are disclosed in U.S. patent application Ser. No. 10/401,031, filed Mar. 28, 2003 which was published as United States Publication No. US 2003/0231546 A1 on Dec. 18, 2003 which is incorporated herein by specific reference (hereinafter “the '031 application).

Returning to FIG. 1, extending through access hole 117 on floor 16 is a mixing shaft 198. As will be discussed and depicted below in greater detail, a mixer is mounted on the first end of mixing shaft 198 within chamber 60. In one embodiment of the present invention, means are provided for selectively raising and lowering mixing shaft 198. By way of example and not by limitation, a frame 168 is centrally disposed below floor 16. In an alternative embodiment, frame 168 can also be mounted to floor 16. Disposed within frame 168 are two spaced apart and vertically oriented guide rods 160 and 161. A carriage 162 is mounted on guide rods 160, 161 so that carriage 162 can move vertically up and down by sliding on guide rods 160, 161. Carriage 162 has a front face 164 and an opposing back face 166 each extending between a top face 170 and an opposing bottom face 172. A locking channel 174 is recessed on front face 164 and vertically extends between top face 170 and bottom face 172.

Mixing shaft 198 is removably disposed within locking channel 174. Mixing shaft 198 and locking channel 174 have complementary interlocking configurations such that when mixing shaft 198 is received within locking channel 174, mixing shaft 198 is fixed within locking channel 174. As a result, vertical reciprocating movement of carriage 162 results in vertical reciprocating movement of mixing shaft 198. By way of example and not by limitation, in the embodiment depicted an annular recess 176 is formed on mixing shaft 198 while a complementary flange 177 outwardly projects within locking channel 174. Flange 177 is received within recess 176 so as to interlock carriage 162 and mixing shaft 198. A cover plate 124 is hingedly mounted on front face 164 of carriage 162 and can be selectively closed over locking channel 174 and then secured in place by a latch 126.

To facilitate reciprocating movement of carriage 162, a wheel 128 is mounted to the back of frame 168 and is rotationally driven by a motor (not shown). A linkage arm 130 is pivotably mounted to the perimeter of wheel 128 and to back face 166 of carriage 162. Accordingly, as wheel 128 is rotated, wheel 128 causes linkage arm 130 to repeatedly raise and lower which in turn causes carriage 162 and mixing shaft 198 to repeatedly raise and lower.

It is appreciated that there are a number of alternative embodiments of the means for selectively raising and lowering mixing shaft 198. By way of example and not by limitation, wheel 128 and linkage arm 130 can be replaced with a number of other forms of drivers such as a pneumatic piston, hydraulic piston, various forms of belt drivers, chain drivers, or gear drivers, or other well known mechanisms that enable repeated raising and lowering of a shaft. It is also appreciated that such drivers can be directly connected to mixing shaft 198 so as to eliminate carriage 162. Other examples of the means for selectively raising and lowering mixing shaft 198 are disclosed in the '031 application.

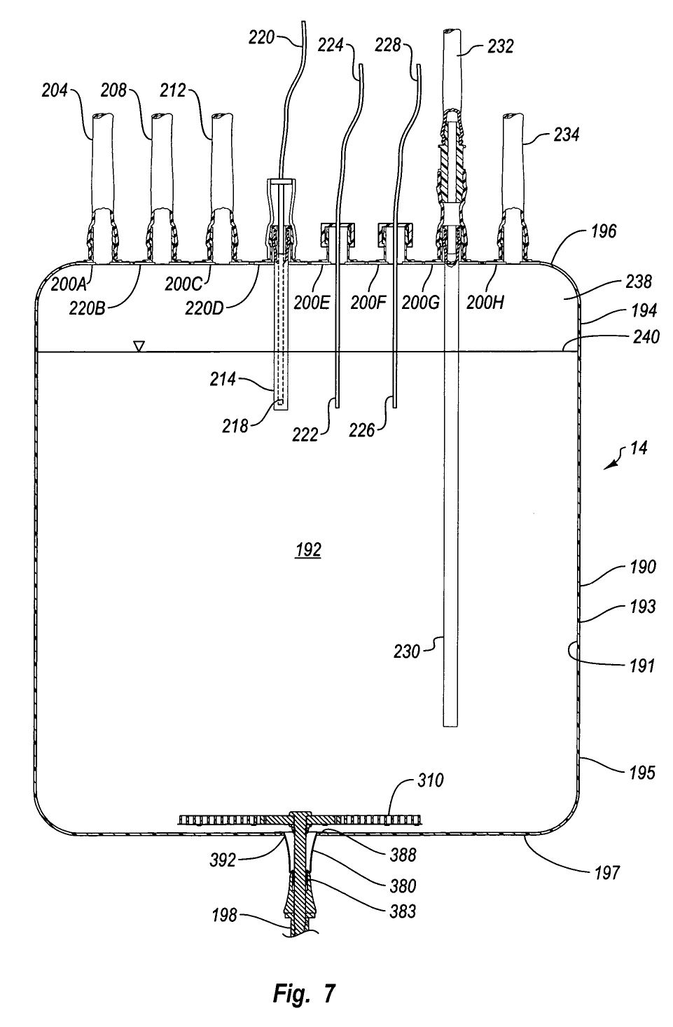

Turning to FIG. 7, container 14 comprises flexible bag-like body 190 having an interior surface 191 that bounds a compartment 192. More specifically, body 190 comprises a side wall 193 that, when body 190 is unfolded, has a substantially circular or polygonal transverse cross section that extends between a first end 194 and an opposing second end 195. First end 194 terminates at a top end wall 196 while second end 195 terminates at a bottom end wall 197.

Body 190 is comprised of a flexible, water impermeable material such as a low-density polyethylene or other polymeric sheets having a thickness in a range between about 0.1 mm to about 5 mm with about 0.2 mm to about 2 mm being more common. Other thicknesses can also be used. The material can be comprised of a single ply material or can comprise two or more layers which are either sealed together or separated to form a double wall container. Where the layers are sealed together, the material can comprise a laminated or extruded material. The laminated material comprises two or more separately formed layers that are subsequently secured together by an adhesive.

The extruded material comprises a single integral sheet that comprises two or more layers of different material that are each separated by a contact layer. All of the layers are simultaneously co-extruded. One example of an extruded material that can be used in the present invention is the HyQ CX3-9 film available from HyClone Laboratories, Inc. out of Logan, Utah. The HyQ CX3-9 film is a three-layer, 9 mil cast film produced in a cGMP facility. The outer layer is a polyester elastomer coextruded with an ultra-low density polyethylene product contact layer. Another example of an extruded material that can be used in the present invention is the HyQ CX5-14 cast film also available from HyClone Laboratories, Inc. The HyQ CX5-14 cast film comprises a polyester elastomer outer layer, an ultra-low density polyethylene contact layer, and an EVOH barrier layer disposed therebetween. In still another example, a multi-web film produced from three independent webs of blown film can be used. The two inner webs are each a 4 mil monolayer polyethylene film (which is referred to by HyClone as the HyQ BM1 film) while the outer barrier web is a 5.5 mil thick 6-layer coextrusion film (which is referred to by HyClone as the HyQ BX6 film).

The material is approved for direct contact with living cells and is capable of maintaining a solution sterile. In such an embodiment, the material can also be sterilizable such as by ionizing radiation. Examples of materials that can be used in different situations are disclosed in U.S. Pat. No. 6,083,587 which issued on Jul. 4, 2000 and U.S. patent application Ser. No. 10/044,636, filed Oct. 19, 2001 which are hereby incorporated by specific reference.

In one embodiment, body 190 comprises a two-dimensional pillow style bag wherein two sheets of material are placed in overlapping relation and the two sheets are bounded together at their peripheries to form internal compartment 192. Alternatively, a single sheet of material can be folded over and seamed around the periphery to form internal compartment 192. In another embodiment, body 190 can be formed from a continuous tubular extrusion of polymeric material that is cut to length and one end seamed closed.

In still other embodiments, body 190 can comprises a three-dimensional bag which not only has an annular side wall but also a two dimensional top end wall 196 and a two dimensional bottom end wall 197. Three dimensional body 190 comprises a plurality of discrete panels, typically three or more and more, commonly four or six. Each panel is substantially identical and comprises a portion of the side wall, top end wall, and bottom end wall of body 190. Corresponding perimeter edges of each panel are seamed. The seams are typically formed using methods known in the art such as heat energies, RF energies, sonics, or other sealing energies.

In alternative embodiments, the panels can be formed in a variety of different patterns. Further disclosure with regard to one method of manufacturing three-dimensional bags is disclosed in U.S. patent application Ser. No. 09/813,351, filed on Mar. 19, 2001 of which the drawings and Detailed Description are hereby incorporated by reference.

It is appreciated that body 190 can be manufactured to have virtually any desired size, shape, and configuration. For example, body 190 can be formed having compartment 192 sized to 30 liters, 100 liters, 250 liters, 500 liters, 750 liters, 1,000 liters, 1,500 liters, 3,000 liters, 5,000 liters, 10,000 liters or other desired volumes. Although body 190 can be any shape, in one embodiment body 190 is specifically configured to be complementary or substantially complementary to chamber 60 of housing 12.

In any embodiment, however, it is desirable that when body 190 is received within chamber 60, body 190 is uniformly supported by floor 16 and side wall 18 of housing 12. Having at least generally uniform support of body 190 by housing 12 helps to preclude failure of body 190 by hydraulic forces applied to body 190 when filled with fluid.

Although in the above discussed embodiment container 14 has a flexible, bag-like configuration, in alternative embodiments it is appreciated that container 14 can comprise any form of collapsible container or rigid container. Container 14 can also be transparent or opaque and can have ultraviolet light inhibitors incorporated therein.

Depicted in FIG. 7, mounted on top end wall 196 are a plurality of ports 200 which are in fluid communication with compartment 192. Each port 200 can serve a different purpose depending on the type of cells or microorganisms being cultured. For example, in the embodiment depicted in FIGS. 1 and 7, a first port 200A is fluid coupled with a media source 202 by a tube 204. Media source 202 can simply comprise a container that houses the growth media. The type or properties of the growth media will depend on the type of cells or microorganisms being cultured. A peristaltic pump or other mechanism can be used to dispense the media from media source 202 into compartment 192.

The term “tube” as used in the specification and appended claims is intended to include conventional flexible hose and tubing which is relatively inexpensive and can be easily replaced, if desired, between the manufacture of different batches or types of culture. The term “tube”, however, is also intended to include rigid piping and other forms of conduits which may be fixed and require sterilization between the manufacture of different batches or types of culture.

Port 200B is fluid coupled with a seed inoculum source 206 by way of a tube 208. Again, seed inoculum source 206 can simply comprise a container housing the seed inoculum. For small scale systems, the seed inoculum can comprise free cells. At later stages along the seed train, the seed inoculum can comprises partially grown cells mixed with a growth media. As previously discussed, the seed inoculum can comprise bacteria, fungi, algae, plant cells, animal cells, protozoans, nematodes, and the like. The cells can be aerobic or anaerobic and adherent or non-adherent. Where adherent cells are being grown, the adherent cells can be pre-attached to micro-carriers in seed inoculum source 206 so as to form a suspension culture once introduced into container 14 and agitated, as described subsequently. Alternatively, the micro-carrier can be introduced into container 14 prior to the addition of the anchorage dependent cells in suspension. The cells will then adhere to the micro-carrier in container 14.

A port 200C is coupled with a pressure regulator 210 by way of an exhaust tube 212. Any conventional pressure regulator can be used that will maintain the sterility of compartment 192. During operation of system 10, pressure regulator 210 is used to release excess gas from compartment 192 while maintaining a positive pressure within compartment 192. The positive pressure helps maintain the sterility of compartment 192. As will be discussed below in greater detail, the positive pressure also helps control the partial pressure of oxygen and other gases within the culture. In one embodiment, the pressure within compartment 192 is maintained within a range between about 0.5 inches of water (1.2×10⁻³ bars) to about 5 inches of water (1.2×10⁻² bars). Other pressures can also be used. If desired, a condenser can also be coupled with pressure regulator 210.

Disposed within a port 200D so as to project down into compartment 192 is a sealed tube 214. Tube 214 is connected to port 200D by use of a dip tube connector 216 or other form of connector. Further disclosure with regard to dip tube connector 216 is disclosed in U.S. Pat. No. 6,086,574, which is incorporated herein by specific reference. A temperature probe 218 is disposed within tube 214 and is used to measure the temperature of the culture within compartment 192. Temperature probe 218 is electrically coupled with a temperature sensor 219 by an electrical wire 220.

Disposed within a port 200E so as to project down into compartment 192 is a dissolved oxygen probe 222. Dissolved oxygen probe 222 is in electrical communication with a dissolved oxygen sensor 223 by way of an electrical wire 224. Probe 222 and sensor 223 function to measure the dissolved oxygen within the culture.

Disposed within a port 200F so as to project down into compartment 192 is a pH probe 226. The pH probe 226 is in electrical communication with a pH sensor 227 by way of an electrical wire 228. Probe 226 and sensor 227 function to measure the pH of the culture.

An elongated dip tube 230 is coupled with a port 200G and extends into compartment 192. Again, a dip tube connector 216 can be used to connect dip tube 230 with port 200G. A dispensing tube 232 is also coupled with port 200G and dip tube 230. Dispensing tube 232 can be used for sampling the culture and removing the final culture.

Finally, a gas tube 234 extends between a port 200F and a gas source 236 such that gas tube 234 is in communication with compartment 192. Container 14 is only partially filled with the growth media and seed inoculum so that a headspace 238 is formed between a top surface 240 of the culture and top end wall 196 of container 14. Gas is delivered from gas source 236 to headspace 238 through gas tube 234 so as to produce a gas overlay within headspace 238. By pressurizing the gas within headspace 238 using pressure regulator 210 and by regulating the concentration or partial pressure of different gases within headspace 238, the partial pressure of the different gases within the culture can also be regulated. That is, the higher pressure of a given gas within headspace 238, the higher concentration of that gas that can be maintained within the culture. Gas source 238 typically provides air that is selectively combined with oxygen, carbon dioxide and/or nitrogen. However, other gases can also be used. The addition of these gases is used to regulate the dissolved oxygen content and pH of the culture.

The gas provided by gas tube 234 can also be used to control the formation of foam on top surface 240 of the culture. Foam can remove desired proteins from the culture. Furthermore, the rupturing of foam bubbles can kill adjacent cells. Thus, in one embodiment a continuous flow of gas can be formed across headspace 238 as a continuous gas stream enters from gas tube 234 and exits out through exhaust tube 212 leading to pressure regulator 210. This continuous flow of gas helps prevent the formation of foam on the surface of the culture.

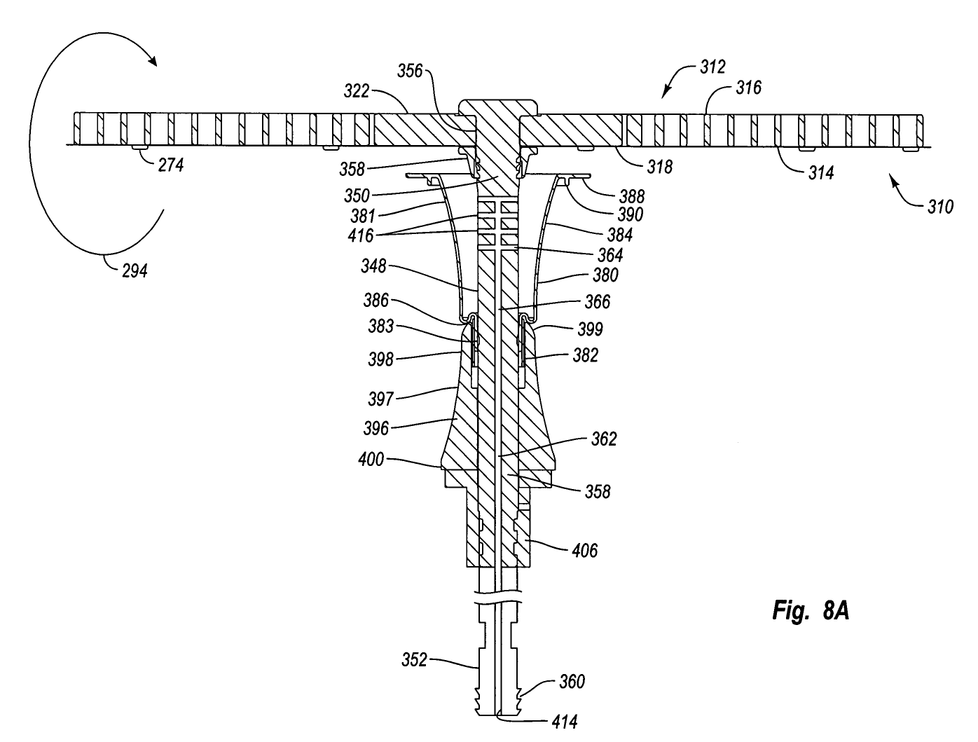

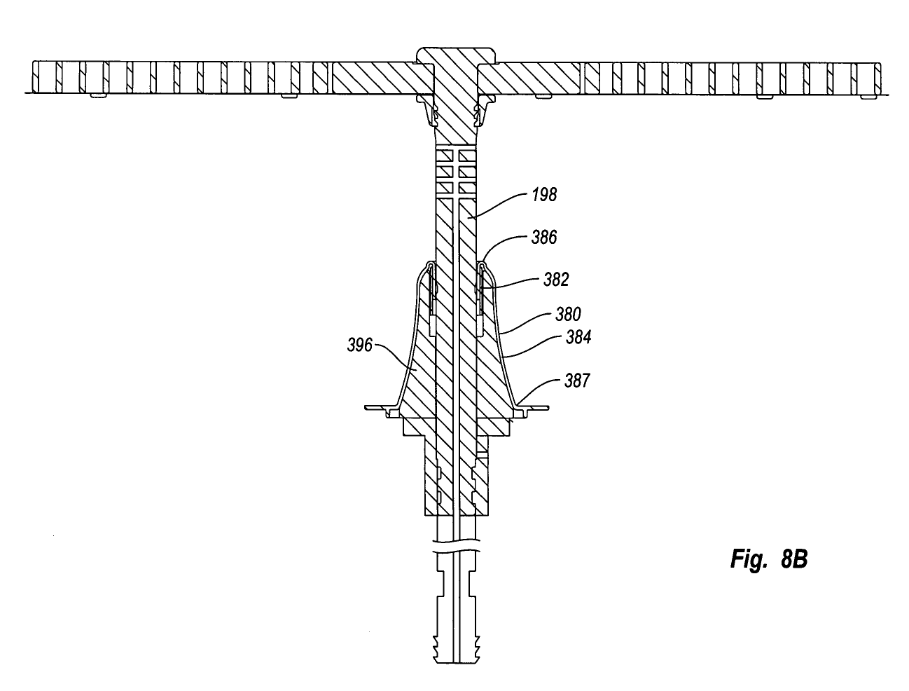

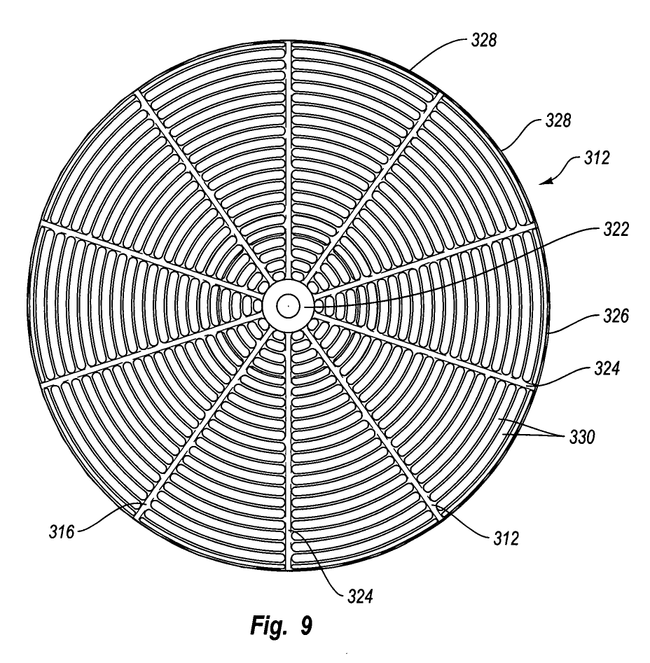

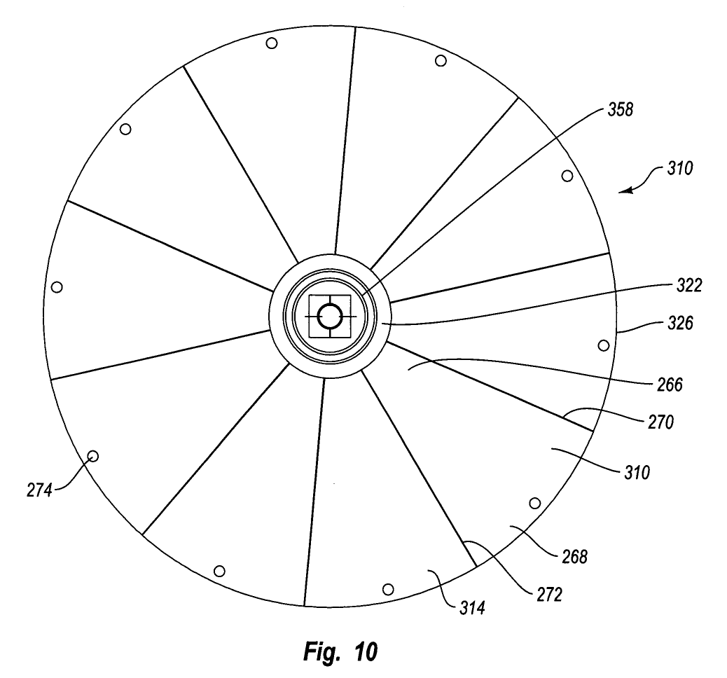

Disposed within compartment 192 at bottom end 195 of container 14 is a vertical mixer 310. Mixer 310 is coupled with mixing shaft 198 that was as previously discussed with regard to FIG. 1. As depicted in FIG. 8A, mixer 310 comprises a base 312 having flaps 314 connected thereto. Base 312 has a substantially circular plate-like configuration having a top surface 316 and an opposing bottom surface 318. As depicted in FIG. 9, base 312 includes an integrally formed central hub 322 and integrally formed struts 324 that radially outwardly project from hub 322 to an outer edge 326. Struts 324 divide base 312 into a plurality of wedge shaped sections 328. Formed within each section 328 so as to extend between top surface 316 and bottom surface 318 are a plurality of fluid openings 330.

Base 312 is typically made of a polymeric material, such as high density polyurethane or polyethylene, but can also be made of metal, composite, or other desired materials. Base 312 can be molded having fluid openings 330 formed thereon. Alternatively, base 312 and/or fluid openings 330 can be cut. In one embodiment, base 312 has a thickness between surfaces 316 and 318 in a range between about 1 cm to about 6 cm with about 2 cm to about 4 cm being more common. Other dimensions can also be used depending on size and use parameters.

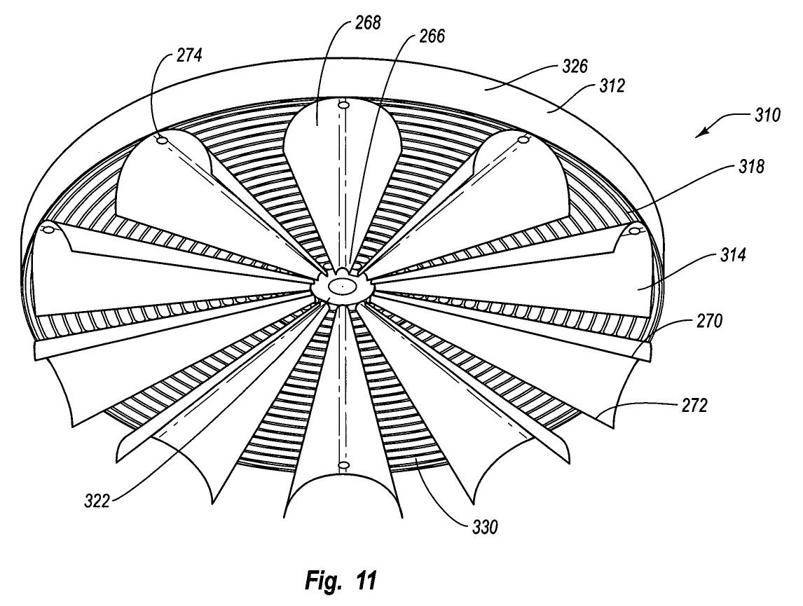

As depicted in FIGS. 10 and 11, a plurality of flexible, wedge shaped flaps 314 are mounted on bottom surface 318 of base 312. Each flap 314 has a narrow lead end 266 disposed against or adjacent to hub 322 and a flared tail end 268 disposed adjacent to outer edge 326. Each flap 314 also comprises opposing diverging sides 270 and 272 that extend from lead end 266 to tail end 268. Each flap 314 is positioned so that a corresponding strut 324 (FIG. 9) extends between lead end 266 and tail end 268 centrally between sides 270 and 272. In one embodiment, each flap 314 is welded, such as by heat, sonic, chemical welding or the like, along the central axis of each flap 314 to the corresponding strut 324. Each flap 314 can also be connected to a corresponding strut 324 by mechanical attachment or other conventional techniques. A tack 274 can also be used to reinforce the connection between each flap 314 and base 312 adjacent to outer edge 326. Each flap 314 is configured to overlay half of each adjacent section 328 with the side edges 270 and 272 of each flap 314 being free to flex. As such, flaps 314 substantially cover bottom surface 318 of base 312.

Mixing of the culture within compartment 192 of container 14 is accomplished by repeatedly raising and lowering mixer 310 within compartment 192. As shown in FIG. 11, as mixer 310 is raised, fluid within compartment 192 passes through fluid openings 330 and pushes against flaps 314 causing sides 270 and 272 of flaps 314 on opposing sides of each strut 324 to downwardly flex, thereby allowing mixer 314 to travel through the culture without substantial disturbance. However, as mixer 314 begins to travel downward, as shown in FIG. 10, the fluid pushes flaps 314 against base 312 so as to block the passage of the culture through fluid openings 330 of mixer 310. As such, downward movement of mixer 310 causes the culture within compartment 192 to flow down, out, up, and around as shown by arrow 294 in FIG. 8A. As the process of raising and lowering mixer 310 is repeated, the swirling motion of the culture caused by mixer 310 results in the culture being uniformly mixed and the cells being uniformly suspended. The mixing process is also performed while minimizing any shear force on culture.

In the present embodiment, it is noted that no ports are formed on bottom end wall 197 of container 14 (FIG. 7). This is because the inventive mixing system can cause the cells to pack and die within such ports. As such, the port are preferably formed on upper end wall 196.

Mixing parameters can be varied based on a variety of parameters such as the size of the mixer, the volume of culture, and the type of culture. For example, the stroke length, i.e., the vertical distance that mixer 310 travels, and the frequency, i.e., the number of times mixer 310 travels the stroke length per unit of time, and the acceleration and deceleration, i.e., the rate at which mixer 310 starts and stops, can each be selectively regulated. The stroke length and frequency can be changed between different uses and can also be changed at different times during a single process. Furthermore, if desired, one or more of the variables can be continually changed during mixing.

In one embodiment, the parameters are set so as to enable thorough mixing of the culture and yet are gentle enough to maintain suspension of the cells for an extended period of time without inducing excess foaming or causing damage to the cells. By way of example and not by limitation, in one embodiment the stroke length is in a range between about 0.1 cm to about 30 cm with about 5 cm to about 20 cm being more common while the frequency is in a range between about 0.1 Hz to about 4 Hz with about 0.5 Hz to about 2 Hz being more common. Other parameter settings, however, can also be used based on the configuration of the mixer and the amount and type of culture.

It is appreciated that the means for mechanically mixing a culture within compartment 192 of container 14 can comprise a variety of modifications or alternative embodiments of mixer 310. For example, in one embodiment mixer 310 can be flipped so that swirling is produced in an opposite direction. Furthermore, flaps 314 are simply functioning as a one-way valve. It is appreciated that there are a variety of alternative ways to form one-way valves on mixer 310. For example, rather than having flexible flaps 314, rigid flaps can be hingedly mounted on mixer 310. Furthermore, pneumatic, hydraulic, mechanical, or electrical switches can be coupled with mixer 310 that selectively open and close one-way valves on mixer 310. In this embodiment, the one-way valves may simply comprise plates that selectively slide to open or close one or more holes extending through mixer 310.

In another alternative embodiment, it is appreciated that mixer 310 can be formed without one-way valves. For example, mixer 310 can comprise a rigid- or flexible plate with no openings. In this embodiment, the plate swirls or otherwise mixes the solution as the plate moves in both directions. In yet another embodiment, the plate can have fixed holes or slots therein to direct movement of the fluid. Likewise, mixer 204 can simply comprise a plurality of fixed fins or vanes which can be configured to either rotate and/or move up and down within container 312 for mixing the solution. In still other embodiments, two or more mixers 310 can be mounted on mixing shaft 198. For example, the mixers 310 can be longitudinally spaced apart along mixing shaft 198. It is likewise noted that mixing shaft 198 can extend through top end wall 196, bottom end wall 197 or side wall 193.

In other embodiments of the means for mixing, mixers can be used that do not operate by being raised and lowered. For example, shaft driven blades and magnetically operated stir bars that rotate within container 14 can be used. Examples of other mixers that can be connected with mixing shaft 198 and/or which can be modified to incorporate gas passages and used with the present invention are disclosed in the '031 application.

As shown in FIG. 8A, mixing shaft 198 is shown connected to mixer 310. Specifically, mixing shaft 198 has an exterior surface 348 extending from a first end 350 to an opposing second end 352. First end 350 terminates at an enlarged head 354. During assembly, second end 352 is advanced down through a hole 356 centrally formed on hub 322 of base 312 until enlarged head 354 rests against top surface 316 of hub 322. A collar 358 is then secured to mixing shaft 198 adjacent to bottom surface 318 of base 312, thereby securing mixer 310 to mixing shaft 198. Collar 358 can also help secure lead end 266 of flaps 314 to base 312. In one embodiment, collar 358 is press fit onto shaft 198. Alternatively, collar 358 can be connected by threaded engagement, crimping or other conventional techniques. It is appreciated that there are a number of other ways in which mixer 310 can be connected to mixing shaft 198. For example, mixing shaft 198 can be integrally molded with base 312 or can be connected by direct threaded engagement, press fit, adhesive, or the like. Still other embodiments for connecting a mixing shaft to a base are disclosed in the '031 application.

Disposed at second end 352 of mixing shaft 198 is a barbed stem 360. Barbed stem 360 is integrally formed with or connected to mixing shaft 198. Stem 360 is adapted to couple with a tube. Centrally extending through mixing shaft 198 from second end 352 to toward first end 350 is a central passage 362. A plurality of spaced apart transverse passages 364 transversely extending through mixing shaft 198 at a location toward first end 350 so as to intersect with central passage 362. Passages 362 and 364 combine to form a gas pathway 366 extending through mixing shaft 198 and having an inlet port 414 and a plurality of sparging ports 416. As depicted in FIG. 1, gas source 236 is coupled with barbed stem 360 of mixing shaft 198 by way of a flexible gas tube 408. Gas tube 408 can be moved up and down as mixing shaft 198 is moved up and down. In this configuration, gas can be passed from gas source 236, though gas tube 408, though gas pathway 366 and into compartment 192 of container 14. The gas is used to sparge the culture within compartment 192 of container 14. In this regard, mixing shaft 198 performs the dual roles of driving mixer 310 and functioning as a sparger. Gas source 236 typically provides air that is selectively combined with oxygen, carbon dioxide and/or nitrogen. Again, other gases can also be used. The addition of these gases is used to regulate the dissolved oxygen content of the culture and to regulate (via dissolved bicarbonate/carbonate content) the pH and pC0₂ of the culture.

The above configuration has a number of unique advantages. Initially, because mixing shaft 198 functions as the sparger, this design eliminates the need for a separate sparger. Typical spargers rest on the floor of conventional bioreactors. Wherein container 14 is a flexible bag, eliminating the need to use conventional spargers is beneficial in that it can be difficult to center and secure a conventional sparger on the floor of a flexible container. Furthermore, such spargers can potentially obstruct the mixing flow within the container. In addition, by sparging though mixing shaft 198, the gas bubbles can be released directly below mixer 310, thereby ensuring uniform and thorough mixing of the gas within the culture. It is appreciated that still other benefits exists. It is noted that some of the above benefits can also be obtained by sparging through a rotating shaft having a propeller or other mixing blades located on the end thereof.

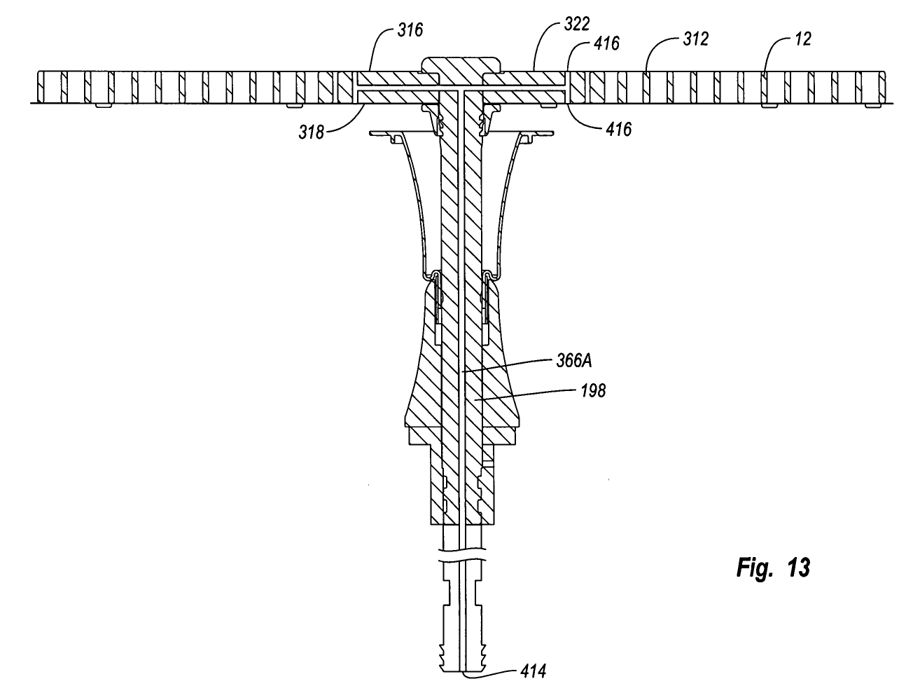

It is appreciated that mixing shaft 198 and mixer 310 can be modified so that gas pathway 366 exits at any desired location on mixing shaft 198 and/or mixer 310. For example, in the embodiment depicted in FIG. 13, a gas pathway 366A is provided which extends along the length of mixing shaft 198, transversely extends out through hub 322, and then exits through sparing ports 416 on top surface 316 and bottom surface 318 of mixer 310. The gas pathway can also be designed to exit at other locations on shaft 198 and/or mixer 310 depending on the position and orientation of mixing shaft 198 and mixer 310.

Returning to FIG. 8A, once mixing shaft 198 is secured to mixer 310, mixing shaft 198 is used for selectively raising and lowering mixer 310 for mixing the culture within compartment 192. The present invention also includes means for enabling mixing shaft 198 to raise and lower mixer 310 within compartment 192 of container 14 while preventing leaking of liquid from compartment 192 of container 14. By way of example and not by limitation, a flexible, tubular diaphragm 380 is provided having a first end 381 and an opposing second end 383. Diaphragm 380 has a tubular sealing portion 382, a tubular flexing portion 384, and an annular joint 386 formed therebetween. Portions 382, 384 and joint 386 are molded as a single integral member. Diaphragm 380 is comprised of an elastomer such styrene butylene styrene blended with a heat sealable plastic such as polyethylene. Other elastomers can also be used.

In the embodiment depicted, sealing portion 382 has a substantially cylindrical configuration with an inside diameter that is substantially equal to the outer diameter of mixing shaft 198. However, because of the flexible nature of diaphragm 380, sealing portion 382 can also be slightly smaller than or larger than mixing shaft 198. Flexing portion 384 radially outwardly flares from joint 386 to first end 381. In one embodiment, flexing portion 384 flares so that the exterior surface of flexing portion 384 has a concave curvature extending along the length thereof. In alternative embodiments, the flare can be substantially linear or produce the exterior surface with a convex curvature.

Encircling and radially outwardly projecting from the free terminal end of flexing portion 384 is an annular flange 388. An annular lip 390 downwardly projects from a bottom surface of flange 388 at a location radially inward from an outer perimeter of flange 388.

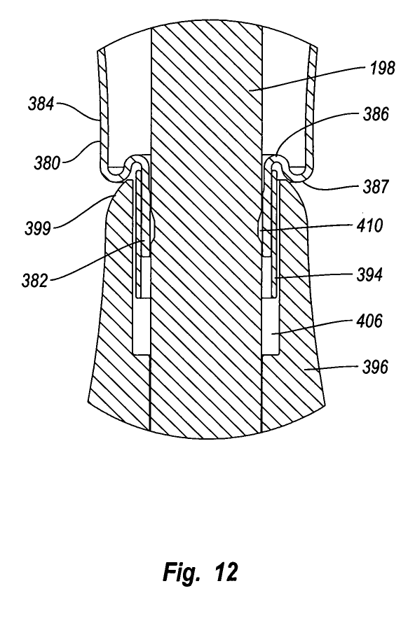

Joint 386 forms an annular shoulder that radially outwardly projects from sealing portion 382 to flexing portion 384. Specifically, as depicted in FIG. 12, joint 386 comprises an annular, substantially U-shaped fold that is integrally molded into diaphragm 380. Thus, joint 386 resiliently returns to the U-shape configuration when diaphragm 380 is in a relaxed state. Joint 386, however, can also be formed with other configurations. As will be discussed below in greater detail, joint 386 helps facilitate vertical displacement of flexing portion 384 as mixing shaft 198 is vertically displaced. In the position depicted in FIG. 12, flexing portion 384 is fully extended above sealing portion 382. As such, an annular substantially U-shaped second fold 387 is formed on flexing portion 384 adjacent to joint 386. Second fold 387, however, is not molded into diaphragm 380 but is simply created due to the positioning of flexing portion 384. As flexing portion 384 is moved downward as discussed below, U-shaped second fold 387 propagates along flexing portion 384.

Depicted in FIG. 7, to secure diaphragm 380 to container 14, a hole 392 is centrally formed through bottom wall 197 of container 14. Second end 383 of diaphragm 380 is advanced down through hole 392 until the bottom surface of flange 388 rests against the interior surface of container 14 bounding hole 392. Container 14 and flange 388 are then secured and sealed together by welding, sealing, or other conventional techniques.

Diaphragm 380 is secured to mixing shaft 198 by a crimp 394. As depicted in FIG. 12, mixing shaft 198 is passed centrally down through diaphragm 380. Crimp 394 is slid over sealing portion 382 so as to encircle sealing portion 382. Crimp 394 is then constricted using a crimping tool so that an annular, liquid tight seal is formed between mixing shaft 198, sealing portion 382, and crimp 394. In one embodiment, crimp 394 is a tube comprised of aluminum. Other metals or crimpable materials can also be used. In still other embodiments, the aluminum tube can be replaced with a tie, clamp, or other mechanisms for constricting sealing portion 382 against mixing shaft 198. To help secure the engagement between mixing shaft 198 and sealing portion 382, an annular detent 410 can be recessed on mixing shaft 198 at a location encircled by sealing portion 382. Sealing portion 382 is thus pressed into detent 410.

Returning to FIG. 8A, also removably mounted on mixing shaft 198 is a tubular guide 396. Guide 396 has an annular exterior surface 397 extending from a first end 398 to an opposing second end 400. First end 398 terminates at an annular tapered nose 399 while a clamp 406 is mounted on second end 400 of guide 396. Exterior surface 397 radially outwardly flares from first end 398 to second end 400 so that the outer diameter at first end 398 is smaller than the outer diameter at second end 400. In the embodiment depicted, exterior surface 397 has a concave curvature extending along the length thereof in a substantially bell shaped contour. In other embodiments, however, exterior surface can be substantially linear or have a convex curvature along the length thereof.

Guide 396 also has a channel 402 centrally extending therethrough. Channel 402 is counter bored at first end 398 so as to form an annular recess 406. During assembly as depicted in FIG. 12, guide 396 is advanced over mixing shaft 198 so that crimp 394 is received within recess 406. Nose 399 is disposed at or adjacent to joint 386 and is typically disposed so that a portion thereof is disposed radially inward of at least a portion of second fold 387. In this position, clamp 406 (FIG. 8A) is secured to mixing shaft 198 so that guide 396 is secured relative to mixing shaft 198. Clamp 406 enables guide 396 to be removed from mixing shaft 198 after use so that guide 396 can be reused. In contrast, mixing shaft 198, mixer 310 and container 14 can be disposable.

During operation, mixing shaft 198 repeatedly raises and lowers due to the movement of carriage 164 as previously discussed with regard to FIG. 1. However, because first end 381 of diaphragm 380 is connected to container 14, first end 381 remains substantially stationary. Accordingly, as mixing shaft 198 raises, flexing portion 384 of diaphragm 380 is inverted as depicted in FIG. 8B. That is, sealing portion 382 advances up into tubular flexing portion 384 while flexing portion 384 rolls or folds outward over guide 396. As flexing portion 384 folds outward, second fold 387 propagates along guide 396. Accordingly, as mixing shaft 198 raises and lowers, diaphragm 380 moves between a first position and a second position as depicted in FIGS. 8A and 8B. The unique configuration of flexing portion 384, joint 386 and guide 396 enables flexing portion 384 to easily roll between the two positions while minimizing kinking or binding of diaphragm 380. As such, diaphragm 380 allows free reciprocating movement of mixing shaft 198 while maximizing the wear life of diaphragm 380. In alternative embodiments, guide 396 can be eliminated. Still other embodiments of the means for enabling mixing shaft 198 to raise and lower mixer 310 within compartment 192 of container 14 while preventing leaking of liquid from compartment 192 of container 14 are disclosed in the '031 application which was previously incorporated by reference.

In one embodiment, container 14, mixing shaft 198 and mixer 310 are manufactured and sold as a disposable unit. During manufacture, a portion of body 190 of container 14 is seamed together as previously discussed. Prior to complete seaming, however, mixer 310 having mixing shaft 198 connected thereto is positioned within compartment 192. Diaphragm 380 is then coupled with container 14 as previously discussed. Once diaphragm 380 is appropriately attached, the remainder of container 14 is seamed closed so as to complete the production. Container 14 is then collapsed and sterilized such as by ionizing radiation or other conventional methods.

Prior to use, the various tubes and probes as previously discussed with regard to FIGS. 1 and 7 are connected to container 14. Connections that need to be sterile are typically made within a laminar hood although some connections can be made prior to sterilization of container 14. In yet other embodiments, the connections can be made after container 14 is disposed within housing 12. Container 14 and the connections can then be sterilized by filling container 14 with a sterilizing gas. Container 14 is positioned within chamber 60 so that second end 352 of mixing shaft 198 is aligned with and passed through central access hole 117 of housing 12. Aligning mixing shaft 198 with access hole 117 can be assisted by the operator reaching through open doorway 57. Mixing shaft 198 is then coupled with carriage 162 as previously discussed. Likewise, gas tube 408 is coupled with mixing shaft 198. Once container 14 is seated within chamber 60, door 25 is closed and locked.

In one embodiment, once container 14 is disposed within housing 12, container 14 is inflated with a gas from gas source 236. This can be accomplished by passing the gas through tube 234 and/or tube 408. Inflating container 14 with a gas makes it easy to ensure that container 14 is properly positioned within housing 12 and that all folds are removed from container 14 so that it is not unduly stressed as container 14 is filled with fluid. Inflation, however, is not necessary, especially for small containers. If desired, a support rack or other supporting structure can be mounted on the top of or above side wall 18. Clamps mounted on the support rack can then be connected to one or more of the ports on container 14 so as to further fix container 14 in place.

Once container 14 is secured within housing 12, media source 202 is used to deliver a growth media into container 14 through tube 204 so as to form headspace 238. The type of growth media depends on the type of cells or microorganisms being cultured. Temperature regulator 49 is then used to heat or cool the growth media within container 14 by heating or cooling the liquid that is passed through fluid compartment 43 in side wall 18 of housing 12. Again, the desired temperature is dependent on the cells or microorganisms being cultured. For example, in one embodiment where a cell line BHK 21 is being cultured, temperature regulator 49 can be used to maintain the temperature of the growth media at a temperature of about 37° C. Other temperatures can also be used. During the heating process, mixer 310 can be activated so that the growth media is heated to a uniform temperature.

Probes 222 and 226 are used to measure the dissolved oxygen and pH of the growth media. Where necessary, gas source 408 introduces varied concentrations of air, carbon dioxide, oxygen, and/or nitrogen through gas tube 408 and mixing shaft 198, so that the growth media has the optimal oxygen concentration and pH for growth of the culture. Again, mixer 310 uniformly disperses the gas throughout the growth media so that the growth media has uniform properties. Once the properties for the growth media has been optimized, the seed inoculum of the cells or microorganisms is introduced from seed source 206 to container 14 through tube 208. Again, where adherent cells are being grown, micro carriers can also be added so as to form a suspension culture.

Once the seed inoculum is added, the resulting culture is continuously mixed by mixer 310 and repeatedly sparged by gas entering through mixing shaft 198. Specifically, as previously discussed, mixer 310 is repeatedly raised and lowered within compartment 192 under various operating parameters specific to the volume and type of culture. In one embodiment where the cell line BHK 21 was being cultured, mixer 310 was operated at 0.5 hertz. Mixer 310 functions to keep the cells uniformly suspended within the growth media so that the cells are uniformly surrounded by the growth media. Mixer 310 also helps prevent unwanted clumping of the cells and helps mix the sparged gas into the culture so that the culture has uniform properties. One of the benefits of mixer 310 is that it is able to efficiently mix both large and relatively small volumes of fluid with minimal shearing forces and while minimizing the formation of foam. The system is thus easily scalable. High shearing forces and the formation of foam can be detrimental to biological materials. Although side wall 18 of housing 20 can be any configuration, such as circular as shown in FIG. 2, it has been discovered that improved mixing properties are obtained if the interior configuration of side wall 18 has a polygonal configuration, such as the hexagonal configuration shown in FIGS. 5 and 6. The polygonal configuration appears to increase turbulent flow which in turn improves mixing.

During the culturing process, the oxygen content and pH of the culture is continually monitor and adjusted by gas source 236. A central processing unit (CPU) 412 can be electrically coupled to each of the components so that necessary adjustments are automatic and continuous. It is also appreciated that other properties of the culture such as glucose, glutamine, lactate, ammonium, and cell density can also be continually monitored and adjusted. Such monitoring can be through probes, sensors, sampling, light scattering techniques, or other methods.

The culture is continually processed until the desired cell density is reached. The culture is then removed from container 14 through dip tube 230 for either end use or further growth in a larger scaled system. Once the grown culture is removed, container 14, mixer 310, shaft 198 and related tubes can be disposed of. Corresponding new, sterile components can then be positioned within housing 12 to repeat the culturing process for a new culture. In one alternative method of use, the present system can also be used in a continuous batch process. Under this process, once a desired cell density is reached, there is a period in which some of the culture is withdrawn and harvested and replaced by fresh media, while cell growth continues. Thus, in the continuous batch process, both the intermediately withdrawn culture and the finally withdrawn culture are each harvested.

In view of the foregoing, system 10 enables the efficient culturing of a culture while minimizing the time, labor and cost associated with cleaning and sterilizing conventional bioreactors. The system also provides for novel mixing which is easily scalable while minimizing foaming. The system also optimizes gas/liquid mass transfer by eliminating conventional spargers while still sparging at preferred locations adjacent to the mixer so as to optimize dispersion of the gas.

The above system and process has largely been described in association with the growth of a biological culture. It is appreciated, however, that the present system for mixing and sparging can also have non-biological uses. For example, media is typically prepared by mixing a powder component with purified water. In conventional processes, the mixing is performed without sparging. In such mixing processes, certain cell culture ingredients (e.g., bicarbonate) are sometimes omitted from the primary powder component and are introduced last. This is because mixing can reduce the bicarbonate concentration. Specifically, during the mixing process, oxygen from the surrounding air is absorbed into the solution. The oxygen alters the pH of some mixtures by striping carbon dioxide from the liquid which reduces the bicarbonate concentration.

By using the present invention, however, the mixing step can be performed while sparging with a gas containing appropriate levels of carbon dioxide. Sparging gases with a pCO₂ correlated with the bicarbonate level of the solution should leave the pH intact. Increasing the carbon dioxide proportion from that level should introduce more bicarbonate and lower the pH. Likewise, decreasing the carbon dioxide proportion from that level should strip some bicarbonate from the liquid and raise the pH. The determination of how much carbon dioxide to add can be measured by using probe 226 and pH sensor 227. Accordingly, by using the present invention, the bicarbonate can be added at the start of the mixing process.

It is appreciated that the inventive system can also be used to process other non-biological fluids where sparing with different gases is used to control oxygen content, pH and other properties.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A system for mixing a liquid solution or suspension comprising:
a collapsible container having an interior surface bounding a compartment, the compartment being adapted to hold a liquid solution or suspension;
a mixer disposed within the compartment of the container;
a shaft having a first end connected to the mixer within the compartment of the container and a second end disposed outside of the compartment, the shaft being selectively movable relative to the container; and
a gas pathway extending from a portion of the shaft disposed outside of the container to a portion of the shaft or mixer disposed within the compartment of the container, the gas pathway extending through at least a portion of the shaft.

2. The system as recited in claim 1, wherein the gas pathway in is communication with a plurality of sparging ports formed on the portion of the shaft or the mixer disposed within the compartment of the container.

3. The system as recited in claim 1, wherein the gas pathway comprises a channel formed within the shaft.

4. The system as recited in claim 1, wherein the shaft is connected to the container so that the shaft can be vertically raised and lowered when the container is substantially filled with a liquid solution or suspension.

5. The system as recited in claim 1, wherein the collapsible container has a top end and an opposing bottom end, the shaft extending through the bottom end of the container, at least one port formed on the top end of the container.

6. The system as recited in claim 5, wherein the bottom end of the bag is absent of any ports formed thereon.

7. The system as recited in claim 1, wherein the compartment of the container is sealed closed.

8. The system as recited in claim 1, wherein the collapsible container comprises a flexible bag comprised of a polymeric material.

9. The system as recited in claim 8, wherein the flexible bag comprises a 3-dimensional bag comprised of a plurality of panels seamed together.

10. The system as recited in claim 1, wherein the mixer is adapted to mix a liquid solution or suspension by being vertically raised and lowered within the liquid solution or suspension.

11. The system as recited in claim 1, wherein the mixer comprises:
a base having a fluid opening extending therethrough; and
a one-way valve positioned adjacent the fluid opening such that when the base moves in a first direction the one-way valve is open so that fluid can flow through the fluid opening and when the base moves in an opposing second direction the one-way valve is closed preventing fluid from flowing through the fluid opening.

12. The system as recited in claim 11, wherein the one-way valve comprises a flexible flap mounted on the base.

13. The mixing system as recited in claim 12, wherein the flexible flap comprises of a polymeric sheet partially welded to the base.

14. The system as recited in claim 1, further comprising means for enabling the shaft to raise and lower the mixer within the compartment of the container while preventing leaking of a liquid solution or suspension from the compartment of the mixing bag.

15. The system as recited in claim 14, wherein the means for enabling the shaft to raise and lower the mixer within the compartment of the container comprises a tubular diaphragm having a first end connected to the container and an opposing second end connected to the mixer or mixing shaft.

16. The system as recited in claim 15, wherein the diaphragm comprises a tubular sealing portion connected to the stem, a tubular flexing portion, and an annular joint formed between the sealing portion and the flexing portion, the joint having a substantially U-shaped transverse cross section.

17. The system as recited in claim 16, wherein the tubular sealing portion, the tubular flexing portion, and an annular joint are molded as a single, integral part.

18. The system as recited in claim 15, wherein at least a portion of the diaphragm is turned inside-out as the shaft raises and lowers within the compartment of the mixing bag.

19. The system as recited in claim 15, further comprising a tubular guide mounted on the shaft below the diaphragm, the guide flaring outward along the length thereof.

20. The system as recited in claim 1, further comprising a substantially rigid housing, the collapsible container being disposed and supported within the housing.

21. The system as recited in claim 1, further comprising means for controlling the temperature of a liquid solution or suspension within the compartment of the collapsible container.

22. The system as recited in claim 21, wherein the means for controlling the temperature of the liquid solution or suspension within the compartment of the collapsible container comprises a substantially rigid housing having an outer jacket, the jacking bounding a fluid pathway with a fluid inlet and a fluid outlet, the collapsible container being disposed and supported within the housing.

23. The system as recited in claim 1, further comprising a culture containing growing cells or microorganisms within the compartment of the container.

24. The system as recited in claim 1, further comprising a gas source coupled with the gas pathway, the gas source providing a gas with controlled levels of oxygen and carbon dioxide to the gas pathway.

25. The system as recited in claim 1, further comprising a pH probe at least partially disposed within the compartment of the container.

26. The system as recited in claim 1, further comprising a dissolved oxygen probe at least partially disposed within the compartment of the container.

27. The system as recited in claim 1, further comprising an exhaust tube coupled with the container, the exhaust tube being coupled with a pressure regulator.

28. A method for mixing a liquid culture containing growing cells or microorganisms, the method comprising:
positioning a collapsible container within a chamber of a substantially rigid housing, the collapsible container bounding a compartment, a mixer and a shaft projecting therefrom being at least partially disposed within the compartment of the container;
feeding one or more components into the compartment of the container to produce a liquid culture containing growing cells or microorganisms;
moving the shaft and mixer within the compartment of the container so as to mix the culture; and
delivering a gas containing a controlled level of oxygen through a sparging port formed on the shaft or the mixer so as to sparge the culture and maintain an oxygen partial pressure in the culture at levels suitable for the continued growth of cells or microorganisms.

29. The method as recited in claim 28, further comprising heating or cooling at least a portion of the housing so as to correspondingly heat or cool the liquid culture within the compartment of the container at levels suitable for the continued growth of the cells or microorganisms.

30. The method as recited in claim 28, wherein the act of moving the shaft and mixer within the compartment comprises repeatedly raising and lowering the shaft and mixer within the compartment.

31. The method as recited in claim 28, wherein the shaft has a gas pathway formed thereon that communicates with the sparging port, the method further comprising coupling a gas source to the shaft so that the gas source communicates with the gas pathway, the gas source providing the gas which comprises controlled levels of oxygen and carbon dioxide to the gas pathway.

32. The method as recited in claim 28, further comprising removing the gas from the compartment of the container at a rate such that the gas maintains a positive gas pressure within the compartment.

33. The method as recited in claim 28, further comprising measuring the dissolved oxygen within the culture.

34. The method as recited in claim 28, further comprising measuring the pH of the culture.

35. A method for mixing a liquid solution, the method comprising:
positioning a collapsible container within a chamber of a substantially rigid housing, the collapsible container bounding a compartment, a mixer and a shaft projecting therefrom being at least partially disposed within the compartment of the container;
feeding two or more components into the compartment of the container, at least one of the components being a liquid;
moving the shaft and mixer within the compartment of the container so as to form a liquid solution;
measuring the pH of the liquid solution or the partial pressure of oxygen within the solution;
delivering a gas containing a controlled level of oxygen or carbon dioxide through a sparging port formed on the shaft or the mixer so as to sparge liquid solution and thereby adjust or regulate the pH or partial pressure of oxygen of the solution.

36. The method as recited in claim 35, wherein the act of moving the shaft and mixer within the compartment comprises repeatedly raising and lowering the shaft and mixer within the compartment.

37. The method as recited in claim 35, wherein the shaft has a gas pathway formed thereon that communicates with the sparging port, the method further comprising coupling a gas source to the shaft so that the gas source communicates with the gas pathway, the gas source providing the gas which comprises controlled levels of oxygen and carbon dioxide to the gas pathway.

38. The method as recited in claim 35, wherein the liquid solution is substantially free to cells or microorganisms.

39. The method as recited in claim 38, wherein the liquid solution comprises a media.

40. The method as recited in claim 38, wherein at least one of the components comprise a powder.
